(19) United States
(12) Patent Application Publication
(10) Pub. No.: US 2010/0240763 A1
(43) Pub. Date: Sep. 23, 2010

(54) Method to Determine and Biomarker for Treatment Efficacy With Ssri, Snri, and Sari Antidepressants

(76) Inventors: Francis S. Lee, New York, NY (US); Zhe-yu Chen, New York, NY (US); Barbara L. Hempstead, New York, NY (US); Kevin G. Bath, New York, NY (US)

Correspondence Address: Irving N Feit;Hoffmann & Baron, 6900 Jericho Turnpike, Syosset, NY, 11791, US

(21) Appl. No.: 12/223,053

(22) PCT Filed: Jan. 19, 2007
(86) PCT No.: PCT/US07/01560

§ 371 Date: Jun. 7, 2010

Related U.S. Application Data
(60) Provisional application No. 60/760,591, filed on Jan. 20, 2006.

(51) Int. Cl. C12Q 1/68; A61K 31/138; A61P 25/24

(52) U.S. Cl. 514/651; 435/6

(57) ABSTRACT

The invention provides a method for determining whether a patient suffering from a condition that is susceptible to treatment with a compound that activates the brain serotonin system is resistant to treatment with the compound. The method comprises observing whether the genome of the patient contains at least one copy of the BDNF allele containing a genetic alteration, and correlating the presence of the allele containing the genetic alteration with patients who are resistant to treatment with the compound. In another embodiment, the method comprises observing whether the patient expresses a BDNF protein containing an amino acid alteration, and correlating the expression of the BDNF protein containing the amino acid alteration with patients who are resistant to treatment with the compound.

This application asserts priority to U.S. Provisional Application Ser. No. 60/760,591 filed on Jan. 20, 2006, the specification of which is hereby incorporated by reference in its entirety.

This invention described in this application was made with funds from the National Institutes of Health, grant number R01 NS052819. The U.S. government has rights in the invention.

### BACKGROUND OF THE INVENTION

Brain-derived neurotrophic growth factor (BDNF) is a protein that is widely expressed in the human brain. The protein plays a critical role in the development and maintenance of neurons in the central nervous system and the peripheral nervous system. For example, BDNF helps to support the survival of existing neurons, and encourage the growth and differentiation of new neurons and synapses.

In the brain, BDNF is active in the hippocampus, cortex, and basal forebrain, These areas are vital to learning, memory, and higher thinking. Previous reports have indicated a possible link between low levels of BDNF and psychiatric conditions such as mood disorders and depression.

Anti-depressants, such as selective serotonin reuptake inhibitors (SSRIs), are typically prescribed to patients suffering from psychiatric conditions, such as depression. However, some patients are resistant to SSRIs. The time period required to determine whether a patient will respond positively to SSRI treatment or be resistant can be costly and lengthy.

Therefore, there is a need for new methods for determining whether a patient will be resistant to treatment with a compound that activates the brain serotonin system.

### SUMMARY OF THE INVENTION

In one embodiment, the invention relates to a method for determining whether a patient suffering from a condition that is susceptible to treatment with a compound that activates the brain serotonin system is resistant to treatment with the compound. The method comprises observing whether the genome of the patient contains at least one copy of the BDNF allele containing a genetic alteration, and correlating the presence of the allele containing the genetic alteration with patients who are resistant to treatment with the compound. In another embodiment, the invention relates to a method for determining whether a patient suffering from a condition that is susceptible to treatment with a compound that activates the brain serotonin system is resistant to treatment with the compound. In another embodiment, the method comprises observing whether the patient expresses a BDNF protein containing an amino acid alteration, and correlating the expression of the BDNF protein containing the amino acid alteration with patients who are resistant to treatment with the compound.

### BRIEF DESCRIPTION OF THE FIGURES

FIG. 1. Nucleotide sequence of wild type BDNF gene.

FIG. 2. GenBank Accession No. NP 001700. Amino acid sequence of wild type BDNF.

FIG. 3. Generation and validation of BDNF_{Met} transgenic mice. (A) Schematic diagram of the strategy used to replace the coding region of the BDNF gene with BDNF_{Met}. The entire coding region is in exon V. For the variant BDNF, a point mutation has been made (G196A) to change the valine in position 66 to methionine. (B) Southern blots of representative embryonic stem cell clones for BDNF_{Met}. Bgl II and Barn HI restriction enzyme digestion and 5′ external probe indicated in (A) were used to detect homologous replacement in the BDNF locus. The 5.6 kilobase (kb) wild type (WT) and 7.2 kb rearranged variant DNA bands are indicated. (C) BDNF ELISA analyses of total BDNF levels from postnatal day 21 (P21) brain lysates from WT (+/+), heterozygous (+/Met), and homozygous (Met/Met) mice, as well as BDNF heterozygous KO mice (+/−) (**P<0.01, Student's t test). (D) Hippocampal-cortical neurons obtained from embryonic day 18 (E18) BDNr^{+/Met} (+/Met), BDNF^{Met/Met} (Met/Met), and WT (+/+) pups were cultured. After 72 hours, media were collected under depolarization (regulated) or basal (constitutive) secretion conditions. Media were then concentrated and analyzed by BDNF ELISA. (*P<0.05, **F′<0.01, Student's t test).

FIG. 4. Altered hippocampal anatomy, and behavior in transgenic BDNF_{Met} mice. (A) Total hippocampal volume estimations were obtained from Nissl-stained sections of adult (P60) hippocampal from WT (+/+), heterozygous (+/Met), homozygous (Met/Met), and heterozygous BDNF KO (+/−) mice by Cavalieri analyses. All results are presented as means±SEM determined from analysis of six mice per genotype (***P<0.001, Student's t test). (B) Examples of Golgi-stained dentate gyrus neurons from P60 WT (+/+), heterozygous (+/Met), homozygous (Met/Met), and heterozygous BDNF KO (+/−) mice. (C) Sholl analyses of dentate gyrus neurons from P60 mice, five mice per genotype, 10 neurons per mouse. All results are presented as means±SEM determined from analysis of five mice per genotype and statistics in comparison with WT controls (*P<0.001). Fear-conditioned learning in adult transgenic BDNF_{Met} mice. WT (+/+), heterozygous (+/Met), homozygous (Met/Met), and heterozygous BDNF (+/−) KO mice were tested in (D) contextual and (E) cue-dependent fear conditioning. The percentage of time spent freezing in each session was quantified. All results are presented as means±SEM determined from analysis of eight mice per genotype (*P<0.05, **P<0.01, Student's t test.

FIG. 5. Anxiety-related behavior in BDNF^{Met/Met} mice in the open field (A and B) and elevated plus maze (C and D). Percentage of time spent in the center (A) and entries into the center (B) in the open field are shown, as well as percentage time spent in the open arm (C) and percentage of open arm entries (D) in the plus maze. All results are presented as means±SEM determined from analysis of eight mice per genotype (**P<0.01).

FIG. 6. Decreased response to long-term fluoxetine in BDNF^{Met/Met} mice in the (A) open-field and (B) novelty-induced hypophagia tests. In the open-field test, percentage of time spent in the center in the absence (H₂O) or presence of fluoxetine (drug) treatment was measured. In the novelty-induced hypophagia test, latency to begin drinking in a novel cage in the absence (H₂O) or presence of fluoxetine (drug) treatment is shown in seconds. All results are presented as means±SEM determined from analysis of eight mice per genotype (*P<0.05, **P<0.01).

### DETAILED DESCRIPTION OF THE INVENTION

The invention provides a method for determining whether a patient suffering from a condition that is susceptible to treatment with a compound that activates the brain serotonin system is resistant to treatment with the compound.

The first step in the method of the present invention is observing whether the genome of the patient contains at least one copy of the brain-derived neurotrophic factor (BDNF) allele containing a genetic alteration.

Brain-derived neurotrophic factor (BDNF) is a member of the neurotrophin family of proteins, a group of highly conserved polypeptide growth factors that also includes nerve growth factor, neurotrophin-3 (NT-3) and neurotrophin-4/5 (NT-4/5). BDNF binds to the TrkB receptor.

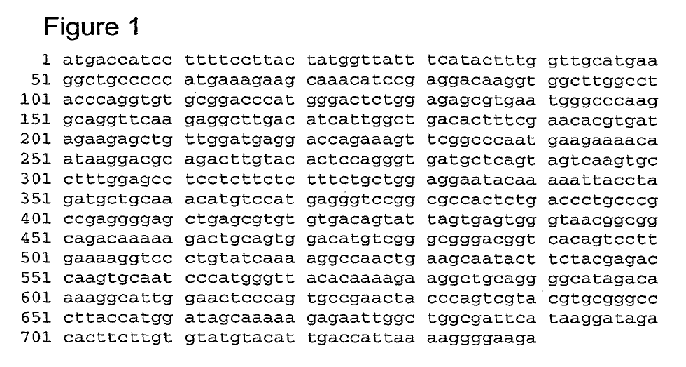

The BDNF gene, which in humans is found on chromosome 11, spans over 40 kB. Typically, the BDNF gene has at least four 5′-exons (exons I, II, III, and IV) that are associated with distinct promoters, and one 3′-exon (exon V). The wild-type BDNF gene comprises a nucleotide coding sequence of pre-pro-BDNF DNA, which is shown in FIG. 1. The nucleotide sequence that encodes the pre-domain, which is also referred to as the signal peptide, comprises the nucleotide sequence beginning at base 1 and ending at base 54 of FIG. 1. The nucleotide sequence that encodes the pro-domain comprises the sequence beginning at base 55 and ending at base 384 of FIG. 1. The nucleotide sequence that encodes the mature domain of the BDNF protein comprises the nucleotide sequence from base 385 to base 741 of FIG. 1.

The genome of a patient generally contains two BDNF alleles. An allele, as used herein, is any of one or more alternative forms of a gene. In an organism, two alleles of a given gene occupy corresponding loci on a pair of homologous chromosomes. For example, two alleles of a BDNF gene occupy corresponding loci on chromosome 11.

The term “genetic alteration,” as used herein, refers to any changes in one or more of the nucleic acid molecules in the nucleotide coding sequence of wild-type BDNF that leads to a change in the amino acid sequence of wild-type BDNF. Accordingly, a BDNF allele that has a nucleotide coding sequence that leads to a change in the amino acid sequence different from the wild-type BDNF constitutes one or more genetic alterations. Examples of genetic alterations include one or more nucleotide additions, deletions, substitutions, etc, and combinations thereof. The genetic variation may, or may not, result in a frame shift.

Wild-type BDNF contains a pre-domain, a pro-domain and a mature domain. The genetic variation may be in any one of these domains, in two of the domains such as in both the pro-domain and the mature domain, or in all three domains.

Nucleotide additions and deletions refer to the addition and deletion, respectively, of one or more nucleotides in the nucleotide sequence of wild-type BDNF. If more than one nucleotide is added or deleted, the additions and deletions can be contiguous or non-contiguous. Any nucleotide (A, T, C, G), and any combination thereof, can be added or deleted. Additions and deletions may result in a frame shift, or may not result in a frame shift.

Nucleotide substitutions refer to the replacement of one or more nucleotide with a different nucleotide. An example of a substitution is a single nucleotide polymorphism.

The genetic alterations can occur at any nucleotide position(s) in the nucleotide sequence of BDNF. For example, the genetic alteration can occur at the beginning, middle or end of the nucleotide sequence. For instance, the genetic alteration(s) can occur anywhere between nucleotides at positions 1 to 1028 of the BDNF nucleotide sequence. Preferably, the genetic alteration occurs in the pre-pro-domain of BDNF, i.e. nucleotides at positions 1-384 of FIG. 1.

A single nucleotide addition, deletion, or substitution within the genome of a person is a genetic alteration, which is herein referred to as a single nucleotide polymorphism (SNP). The standard nomenclature for representing a SNP by those skilled in the art is by a reference SNP number (rs#).

In one embodiment, the genetic alteration is a SNP in which the G nucleotide at position 196 of wild-type BDNF as shown in FIG. 1 is substituted with the nucleotide A. Such SNP is referred to as “G196A.” The reference SNP number for G196A is rs6265.

In another embodiment, the genetic alteration is an SNP in which the C nucleotide at position 5 of wild-type BDNF is substituted with the nucleotide T. Such SNP is referred to as “C5T” (rs#8192466).

In yet another embodiment, the genetic alteration is an SNP in which the G nucleotide at position 225 of wild-type BDNF is substituted with the nucleotide T. Such SNP is referred to as “G225T” (rs#1048218).

In another embodiment, the genetic alteration is an SNP is which the G nucleotide at position 374 of wild-type BDNF is substituted with the nucleotide T. Such SNP is referred to as “G374T” (rs#1048220).

In a further embodiment, the genetic alteration is an SNP in which the G nucleotide at position 380 of wild-type BDNF is substituted with the nucleotide T. Such SNP is referred to as G380T (rs#1048221).

In yet a further embodiment, the genetic alteration can be a combination of any of the SNPs described above.

A genetic alteration may occur within one copy or both copies of a BDNF allele. A person's homologous chromosomes may comprise identical alleles of the BDNF gene at corresponding loci, in which case, the person's BDNF genotype is homozygous for the BDNF gene. Alternatively, a person's homologous chromosomes may not comprise identical alleles of the BDNF gene at corresponding loci, in which case, the person's BDNF genotype is heterozygous for the BDNF gene.

The patient's BDNF genotype can be homozygous or heterozygous for any genetic alteration, such as those mentioned above. For example, in one embodiment, the BDNF genotype is homozygous for G196A. In another embodiment, the BDNF genotype is heterozygous for G196A.

The observation of an allele containing a genetic alteration can be made by any method known to those skilled in the art. Suitable methods are provided in the “General Methods” section below.

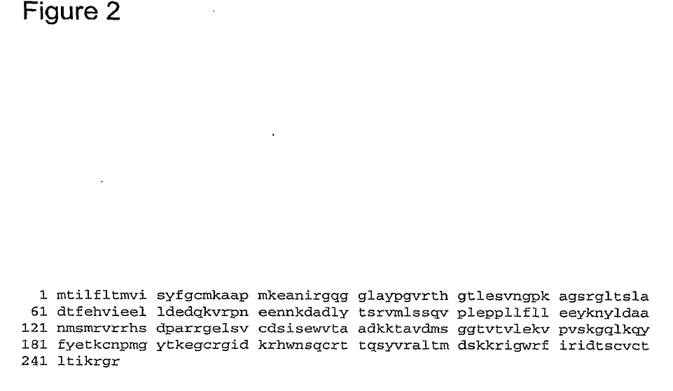

In another embodiment, the method comprises observing expression of a BDNF protein containing an amino acid alteration. The amino acid sequence of wild-type BDNF protein is shown in FIG. 2. The amino acid sequence of the wild-type BDNF pre-domain (signal peptide) comprises the sequence beginning at amino acid residue 1 and ending at residue 18 of FIG. 2. The amino acid sequence of wild-type BDNF pro-domain comprises the sequence beginning at amino acid residue 19 and ending at residue 128 of FIG. 2. The amino acid sequence of wild-type BDNF mature domain comprises the sequence beginning at amino acid residue 129 and ending at residue 247 of FIG. 2.

Typically, the pre-pro-domain of the BDNF protein is cleaved from the mature domain. BDNF protein includes the complete (i.e., uncleaved) protein, cleaved protein, and precursor protein. In wild-type BDNF protein, the complete protein is amino acid residues 1 to 247 of FIG. 2, the cleaved protein is amino acid residues 128 to 247 of FIG. 2), and the precursor protein is amino acid residues 1 to 127 of FIG. 2.

In one embodiment, the method comprises observing whether the patient expresses an uncleaved BDNF protein. In another embodiment, the method comprises observing whether the patient expresses a cleaved BDNF protein. In another embodiment, the method comprises observing whether the patient expresses a precursor BDNF protein.

The term “amino acid alteration” refers to any changes in the amino acid sequence of wild-type BDNF protein. Thus, BDNF proteins that contain an amino acid alteration will have a different amino acid sequence than wild-type BDNF protein. Examples of amino acid alterations include one or more amino acid additions, deletions, substitutions, etc. and combinations thereof, e.g. any of the amino acid alterations caused by the genetic alterations described above.

In one embodiment, the amino acid alteration is a substitution of the amino acid valine at position 66 of wild-type BDNF, shown in FIG. 2, with methionine. The nomenclature for representing such an alteration is known by those skilled in the art as val66met.

In another embodiment, the amino acid alteration is a substitution of the amino acid threonine at position 2 of wild-type BDNF with isoleucine. The nomenclature for representing such an alteration is known by those skilled in the art as thr2ile.

In yet another embodiment, the amino acid alteration is a substitution of the amino acid glutamine at position 75 of wild-type BDNF with the amino acid histidine. The nomenclature for representing such an alteration is known by those skilled in the art as gln75his.

In a further embodiment, the amino acid alteration is a substitution of the amino acid arginine at position 125 of wild-type BDNF with the amino acid methionine. The nomenclature for representing such an alteration is known by those skilled in the art as arg125met.

In yet a further embodiment, the amino acid alteration is a substitution of the amino acid arginine at position 127 of wild-type BDNF with the amino acid leucine The nomenclature for representing such an alteration is known by those skilled in the art as arg127leu.

The observation of expression of a BDNF protein containing an amino acid alteration can be made by any method known to those skilled in the art. Suitable methods are provided in the “General Methods” section below.

The presence of a BDNF allele containing a genetic alteration in a patient, or the presence of a BDNF protein containing an amino acid alteration, is correlated with patients who are resistant to treatment with compounds that activate the brain serotonin system. The term “correlate” refers to relating the presence of a BDNF allele containing a genetic alteration, or the presence of a BDNF protein, with the likelihood that the patient is resistant to treatment with compounds that activate the brain serotonin system.

The correlation step can be carried out without the need for a qualified medical practitioner. For example, a laboratory technician can perform the correlation step.

Patients who are resistant to treatment with compounds that activate the brain serotonin system do not obtain as clinically beneficial an effect in their condition from compounds that activate the brain serotonin system as do patients that are homozygous for wild-type BDNF.

Patients who are homozygous for a genetic alteration generally have little or no significant beneficial effect from compounds that activate the brain serotonin system. For example, compounds that activate the brain serotonin system do not significantly alleviate the patient's condition.

Alternatively, patients who are heterozygous for a genetic alteration generally have a decreased effect from the compounds as compared to patients who are homozygous for the wild-type BDNF genotype. For example, the effect may be decreased by at least about 25%, 50% or 75% with respect to patients who are wild-type homozygous.

Any method known to those of ordinary skill in the art can be employed to determine whether the compounds that activate the brain serotonin system have a decreased effect on a patient's condition. For example, a rating scale can be utilized to score the severity of a psychiatric disorder. The patient is then monitored to determine the chronological effect of such a compound. Examples of such rating scales include the Hamilton Rating Scale for Depression (HAM-D), Emotional State Questionnaire or Global Clinical Impression Scale.

The information that a patient is resistant to compounds that activate the brain serotonin system is very useful. For example, medical personnel may prescribe treatments for such patients other than the administration of such compounds. Examples of such other treatments include vagus nerve stimulation, electroconvulsive therapy, transcranial magnetic stimulation, lithium, gamma-amino butyric acid agonists (e.g., pregabalin (Lyrica™)), and dopamine specific agonists (e.g., buproprion (Wellbutrin™)).

### Compound

The compound can be any compound that activates the brain serotonin system. Serotonin is a neurotransmitter generally secreted by nerve cells. Typically, some of the secreted serotonin is reabsorbed by the cell that secreted it. Such reabsorption is called serotonin reuptake.

Activation of the brain serotonin system by the compound increases the levels of serotonin in the brain. The compound can activate the brain serotonin system by any method known to those in the art. For example, the compound can increase secretion levels by increasing the secretion of serotonin or inhibiting the reuptake of serotonin. Examples of compounds that activate the brain serotonin system include tricyclic antidepressants, selective serotonin reuptake inhibitors (SSRI), selective norepinephrine reuptake inhibitors (SNRI), and serotonin antagonist and reuptake inhibitors (SARI).

Examples of tricyclic antidepressants include amitriptyline (Elavil™), clomipramine (Anafranil™), desipramine (Norpramin™), doxepin (Sinequan™), imipramine (Tofranil™), nortriptyline (Pamelor™), and protriptyline (Vivactil™).

Examples of SSRI include fluoxetine (Prozac™), fluvoxamine (Luvox™), paroxetine (Paxil™), sertaline (Zoloft™), citalopram (Celexa™), and escitalopram oxalate (Lexapro™).

Examples of SNRI include duloxetine (Cymbalta™) and venlafaxine (Effexor™).

Examples of SARI include mirtazapine (Remeron™), nefazodone (Serzone™), and desyrel (Trazodone™).

### Patient

The patient is a human who suffers from any condition that is susceptible to treatment with a compound that activates the brain serotonin system. The patient is generally diagnosed with the condition by skilled artisans, such as a physician (e.g., psychiatrist) or clinician.

The conditions that are susceptible to treatment with a compound that activates the brain serotonin system include any medical disorder. The medical disorder may be a psychiatric disorder.

Examples of psychiatric disorders include affective disorders such as depression (e.g., major depression), bipolar disorder, dysthymia, anxiety disorder (e.g., generalized anxiety disorder, panic disorder, obsessive compulsive disorder, post-traumatic stress disorder, social phobia), and premenstrual dysphoric disorder.

Examples of psychiatric disorders also include eating disorders such as bulimia nervosa and anorexia.

The medical disorder may also include chronic pain. Examples of chronic pain include diabetic neuropathy and postherpetic neuralgia.

The methods of the invention described herein can be employed for patients of any ethnic populations. Examples of ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

### General Methods

To observe whether the genome of a patient contains at least one copy of the BDNF allele containing a genetic alteration, a sample containing the patient's DNA is obtained. Examples of such samples include blood, salvia, urine and epithelial cells.

The sample can be obtained by any method known to those in the art. Suitable methods include, for example, venous puncture of a vein to obtain a blood sample and cheek cell scraping to obtain a buccal sample.

DNA can be isolated from the sample by any method known to those in the art. For example, commercial kits, such as the QIAGEN System (QIAmp DNA Blood Midi Kit, Hilder, Germany) can be used to isolate DNA.

The DNA is optionally amplified by methods known in the art. One suitable method is the polymerase chain reaction (PCR) method described by Saiki et al., Science 239:487 (1988), U.S. Pat. No. 4,683,195 and Sambrook et al. (Eds.), Molecular Cloning, Third Edition, Cold Spring Harbor Laboratory Press, Cold Spring Harbor, N.Y. (2001). For example, oligonucleotide primers complementary to a nucleotide sequence flanking and/or present at the site of the genetic alteration of the allele can be used to amplify the allele.

The isolated DNA is used to determine whether an allele containing a genetic alteration is present in the sample. The presence of an allele containing a genetic alteration can be determined by any method known to those skilled in the art. One method is to sequence the isolated DNA and compare the sequence to that of wild-type BDNF.

Alternative methods include, for example, use of nucleic acid probes and polymerase chain reaction (PCR). Methods for making and using nucleic acid probes are well documented in the art. For example, see Keller G H and Manak M M, DNA Probes, 2^{nd} ed., Macmillan Publishers Ltd., England (1991) and Hames B D and Higgins S J, eds., Gene Probes I and Gene Probes II, IRL Press, Oxford (1995).

For example, methods for distinguishing a wild-type allele from an allele containing a single nucleotide change are described in PCT Application WO 87/07646. The methods disclosed in PCT Application WO 87/07646 are incorporated herein by reference.

Briefly, oligonucleotides containing either the wild-type or an allele containing a genetic alteration are hybridized under stringent conditions to dried agarose gels containing target RNA or DNA digested with an appropriate restriction endonuclease. An example of suitable stringent conditions includes a temperature of two or more degrees below the calculated Tₘ of a perfect duplex. The oligonucleotide probe hybridizes to the target DNA or RNA detectably better when the probe and the target are perfectly complementary.

A particularly convenient method for assaying a single point mutation by means of oligonucleotides is described in Segev, PCT Application WO 90/01069. The methods disclosed in PCT Application WO 90/01069 are hereby incorporated by reference.

Briefly, two oligonucleotide probes for a wild-type and an allele containing a genetic alteration being assayed are prepared. Each oligonucleotide probe is complementary to a sequence that straddles the nucleotides at the site of the genetic alteration. Thus, a gap is created between the two hybridized probes.

The gap is filled with a mixture of a polymerase, a ligase, and the nucleotide complementary to that at the position to form a ligated oligonucleotide product. Either of the oligonucleotides or the nucleotide filling the gap may be labelled by methods known in the art.

The ligated oligonucleotide product can be amplified by denaturing it from the target, hybridizing it to additional oligonucleotide complement pairs, and filling the gap again, this time with the complement of the nucleotide that filled the gap in the first step.

The oligonucleotide product can be separated by size and the label is detected by methods known in the art.

Alleles containing a genetic alteration may also be detected if they create or abolish restriction sites; see Baker et al, Science 244, 217-221 (1989). Some additional examples of the use of restriction analysis to assay point mutations are given in Weinberg et al, U.S. Pat. No. 4,786,718 and Sands, M. S. and Birkenmeier, E. H., Proc. Natl. Acad. Sci. USA 90:6567-6571 (1993).

For example, point mutations can be detected by means of single-strand conformation analysis of polymerase chain reaction products (PCR-SSCP). This method is described in Orita, M. et al., Proc. Natl. Acad. Sci. USA 86:2766-2770 (1989), Suzuki, Y. et al., Oncogene 5:1037-1043 (1990), and Sarkar, F. H. et al., Diagn. Mol. Pathol. 4:266-273 (1995).

Some additional methods for distinguishing a wild-type allele and allele containing a genetic alteration are described by De Ley et al., J. Bacteriol. 101:738-754 (1970); Wood et al., Proc. Natl. Acad. USA 82:1585-1588 (1985); Myers et al., Nature 313:495-497 (1985); and Myers et al., Science 230:1242-1246 (1985).]. See also U.S. Patent Application Publication No. 2005/0014170, which discloses assays for observing BDNF genotypes, the specification of which is hereby incorporated by reference.

To observe whether the patient expresses a BDNF protein containing an amino acid alteration, a sample containing protein is obtained. The sample can be any sample which contains protein. Examples of such samples include blood and spinal fluid. The sample can be obtained by any method known to those in the art.

Protein can be isolated from the sample by any method known to those in the art. For example, commercial kits, such as the Mono Q ion exchange chromatography (Amersham Biosciences, Piscataway, N.J.) can be used to isolate the protein.

The protein can be used, for example, to generate antibodies. The antibody may be polyclonal or monoclonal. Polyclonal antibodies can be isolated from mammals that have been inoculated with the protein in accordance with methods known in the art.

Briefly, polyclonal antibodies may be produced by injecting a host mammal, such as a rabbit, mouse, rat, or goat, with the protein or fragment thereof capable of producing antibodies that distinguish between proteins containing amino acid alterations and wild-type protein. The peptide or peptide fragment injected may contain the wild-type sequence or the sequence containing the amino acid alteration. Sera from the mammal are extracted and screened to obtain polyclonal antibodies that are specific to the peptide or peptide fragment.

The antibodies are preferably monoclonal. Monoclonal antibodies may be produced by methods known in the art. These methods include the immunological method described by Kohler and Milstein in Nature 256, 495-497 (1975) and by Campbell in “Monoclonal Antibody Technology, The Production and Characterization of Rodent and Human Hybridomas” in Burdon et al., Eds, Laboratory Techniques in Biochemistry and Molecular Biology, Volume 13, Elsevier Science Publishers, Amsterdam (1985); as well as the recombinant DNA method described by Huse et al. in Science 246, 1275-1281 (1989).

In order to produce monoclonal antibodies, a host mammal is inoculated with a peptide or peptide fragment as described above, and then boosted. Spleens are collected from inoculated mammals a few days after the final boost. Cell suspensions from the spleens are fused with a tumor cell in accordance with the general method described by Kohler and Milstein in Nature 256, 495-497 (1975). See also Campbell, “Monoclonal Antibody Technology, The Production and Characterization of Rodent and Human Hybridomas” in Burdon et al., Eds, Laboratory Techniques in Biochemistry and Molecular Biology, Volume 13, Elsevier Science Publishers, Amsterdam (1985). In order to be useful, a peptide fragment must contain sufficient amino acid residues to define the epitope of the molecule being detected (e.g., distinguish between wild-type protein and proteins containing amino acid alterations).

The antibodies can, for example, be used to observe the presence of BDNF proteins containing amino acid alterations. Suitable methods include, for example, a western blot and an ELISA assay.

### EXAMPLES

### Example 1

### Generation of Transgenic Mice in which BDNF (Val66Met) is Endogenously Expressed

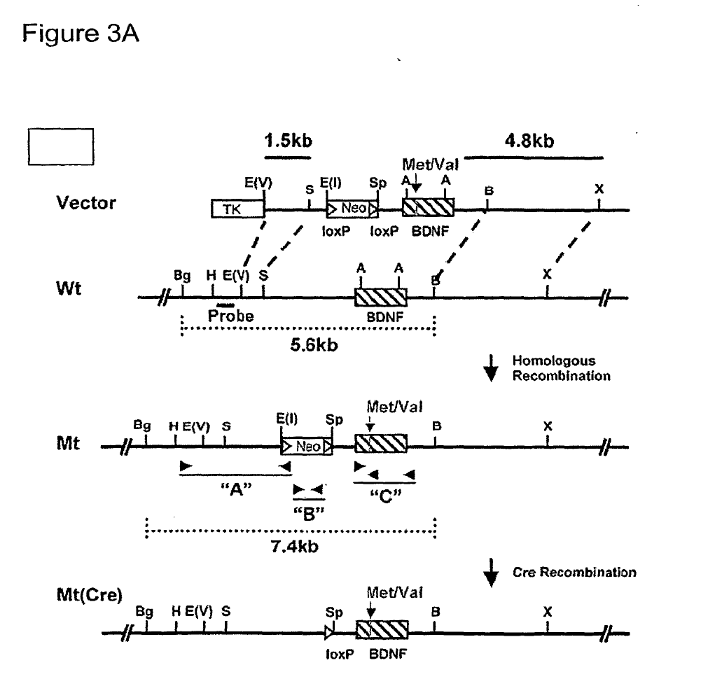

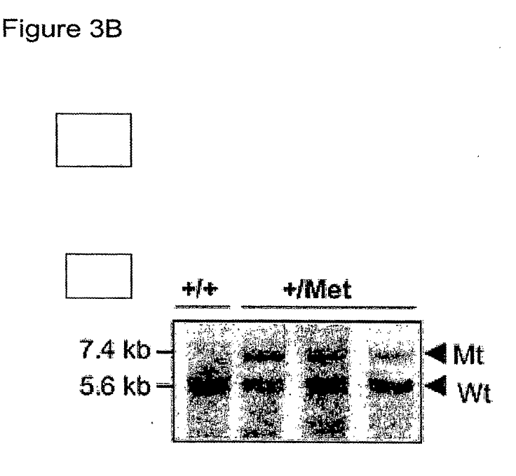

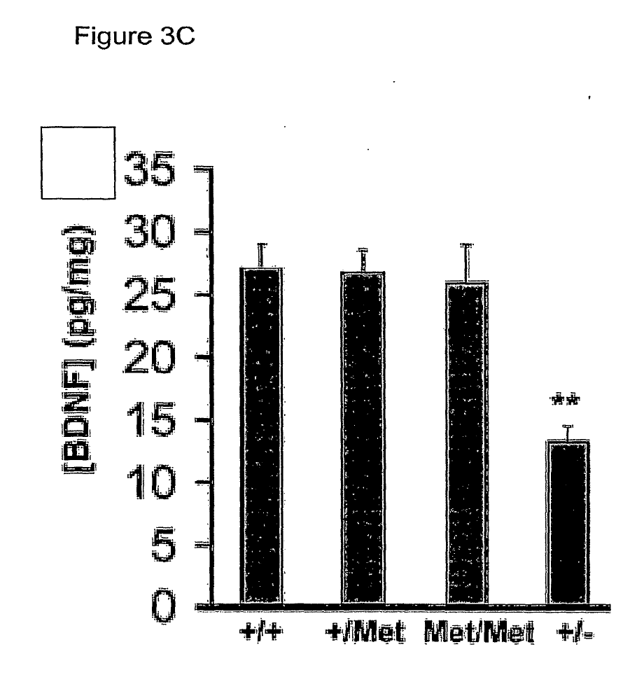

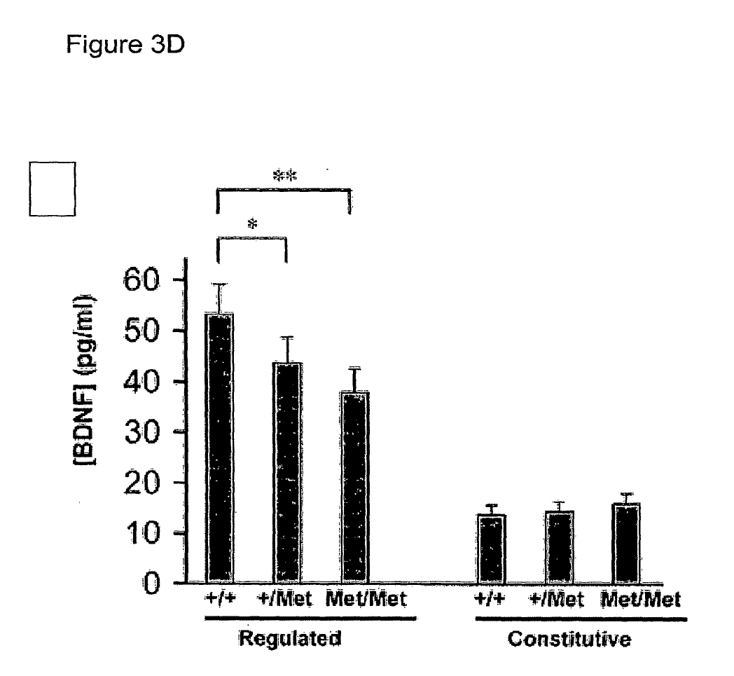

To generate a transgenic mouse in which BDNF (Met) is endogenously expressed, a BDNF (Met) knock-in allele in which transcription of BDNF (Met) is regulated by endogenous BDNF promoters (FIGS. 3A and 3B) was designed. Heterozygous BDNF^{+/Met} mice were intercrossed to yield BDNF^{+/+}, BDNF^{+/Met}, and BDNF^{Met/Met} offspring at Mendelian rates. Brain lysates from BDNF^{+/Met} and BDNF^{Met/Met} mice showed comparable levels of BDNF as that of wild-type controls (FIG. 3C).

To assess whether there were global or selective defects in BDNF (Met) secretion, hippocampal-cortical neurons were obtained from BDNF^{Met/Met}, BDNF^{+/Met} and wild type embryos. Secretion studies were performed, and BDNF in the resultant media was measured by enzyme-linked immunosorbent assay. There was no difference in constitutive secretion from either BDNF^{+/Met} or BDNF^{Met/Met} neurons (FIG. 3D). A significant decrease in regulated secretion from both BDNF^{+/Met} (18±2% decrease, P<0.01) and BDNF^{Met/Met} (29±3% decrease, P<0.01) neurons. As the majority of BDNF is released from the regulated secretory pathway in neurons, impaired regulated secretion (29±3%) from BDNF^{Met/Met} neurons represents a significant decrease in available BDNF.

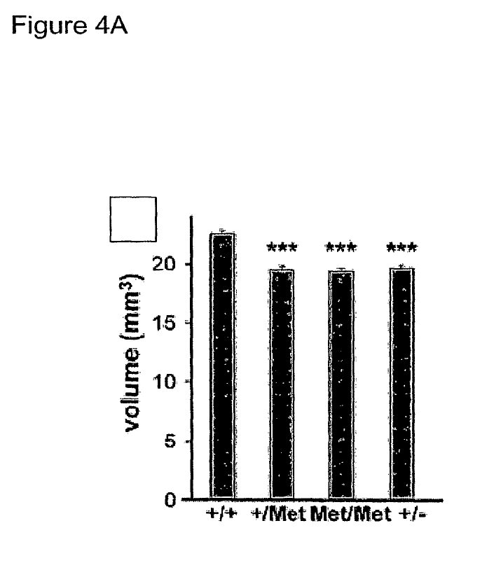

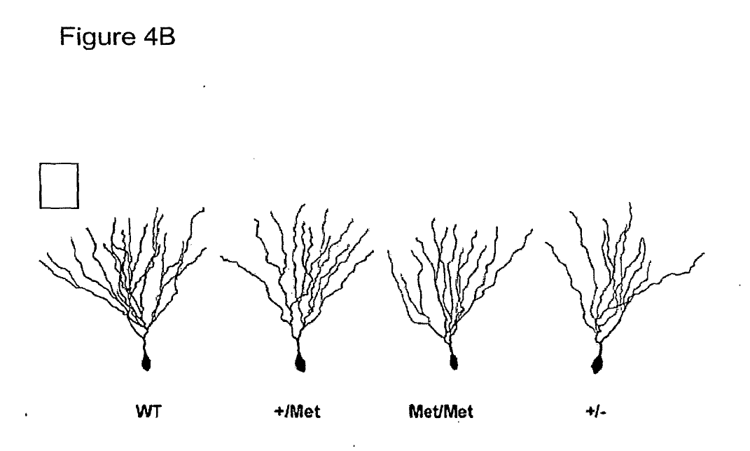

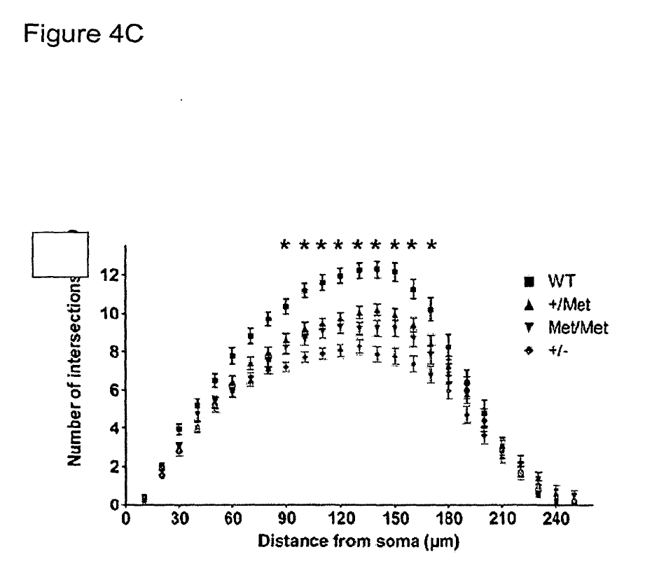

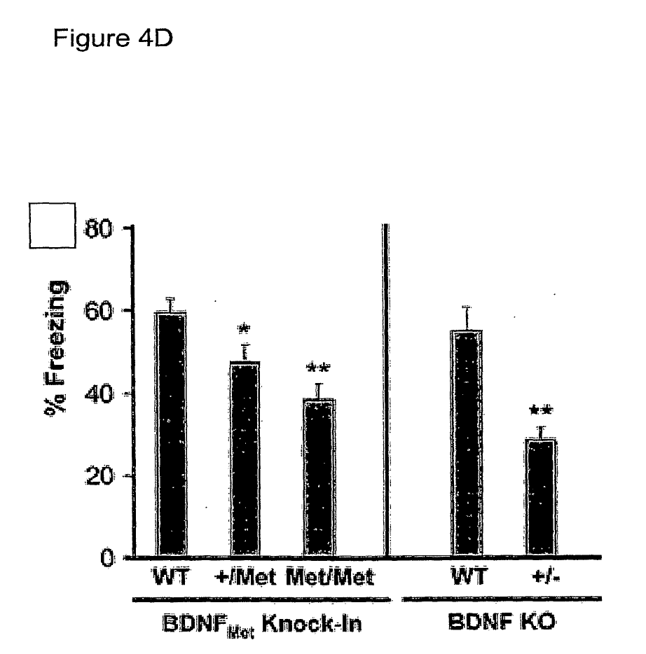

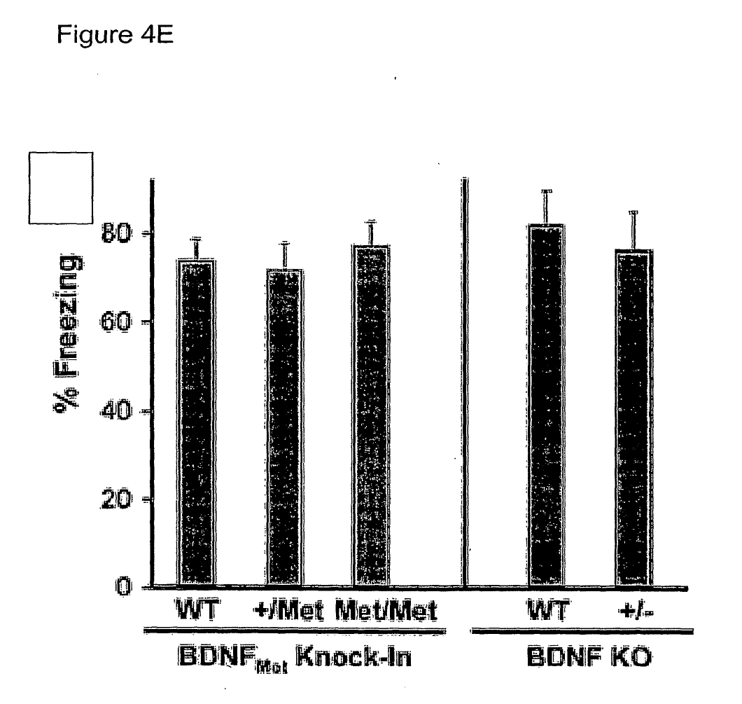

To assess an alteration associated with the Met allele in humans (decrease hippocampal, volume), BDNF_{Met} mice were histologically prepared for stereologic hippocampal volume estimation from Nissl-stained sections. Using Cavalieri volume estimation, a significant decrease in hippocampal volume of 13.7±0.7% and 14.4±0.7% for BDNF^{+/Met or} BDNF^{Met/Met} mice, respectively, as compared with wild-type mice (FIG. 4A). This volume decrease was also comparable to the 13.8±0.6% decrease in the heterozygous BDNF knock-out (BDNF^{+/-}) mice (FIG. 4A). The striatal volume was also measured because in human studies, this structure has not been reported to be altered by the BDNF_{Met} polymorphism. No alteration in mouse striatal volumes across genotypes was found.

Golgi staining was used to visualize individual dentate gyrus neurons. At 8 weeks of age, there was no difference in cell soma area between BDNF^{+/Met} and BDNF^{Met/Met} mice and their wild-type controls. Dendritic complexity in these same neurons were then analyzed (FIG. 4B). Sholl analysis revealed a decrease in dendritic arbor complexity at 90 μM and greater distances from the soma in BDNF^{+/Met} and BDNF^{Met/Met} mice (FIG. 4C). Fractal dimension analysis was to quantify how completely a neuron fills its dendritic field. There was s significant decrease in dendritic complexity in dentate gyrus neurons from BDNF^{+/Met} and BDNF^{Met/Met} mice.

In humans, the other major alteration associated with the BDNF_{Met} allele is impairment in hippocampus-dependent memory. A test was performed that selectively assesses hippocampus- and amygdala-dependent learning: fear conditioning. BDNF^{+/Met} and BDNF^{Met/Met} mice showed significantly less context-dependent memory than wild-type mice (FIG. 4D). In contrast, there was no difference in cue-dependent fear conditioning (FIG. 4E). The degree of memory impairment was related to the number of alleles of BDNF_{Met} (FIG. 4D). BDNF^{Met/Met} mice displayed other behavioral abnormalities similar to BDNF^{+/-} mice, such as intermale aggressiveness. BDNF^{Met/Met} mice also displayed elevated body weight, which was first evident at 2 months of age, similar to BDNF^{+/-} mice. BDNF BDNF^{+/Met}, and BDNF^{Met/Met} mice had no significant alterations in locomotor activity.

### Example 2

### Effect of the Serotonin Reuptake Inhibitor, Fluoxetine, on BDNF (Val66Met) Transgenic Mice

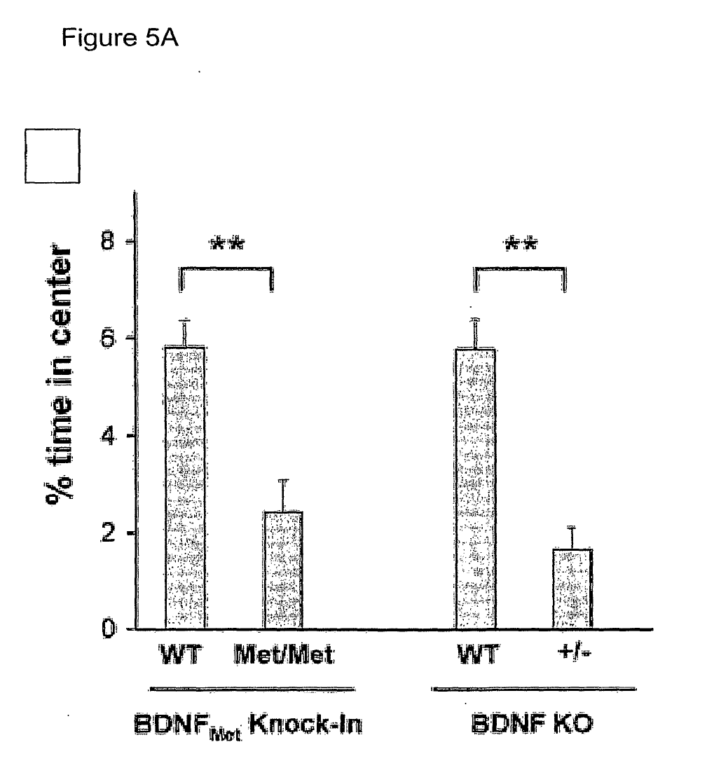

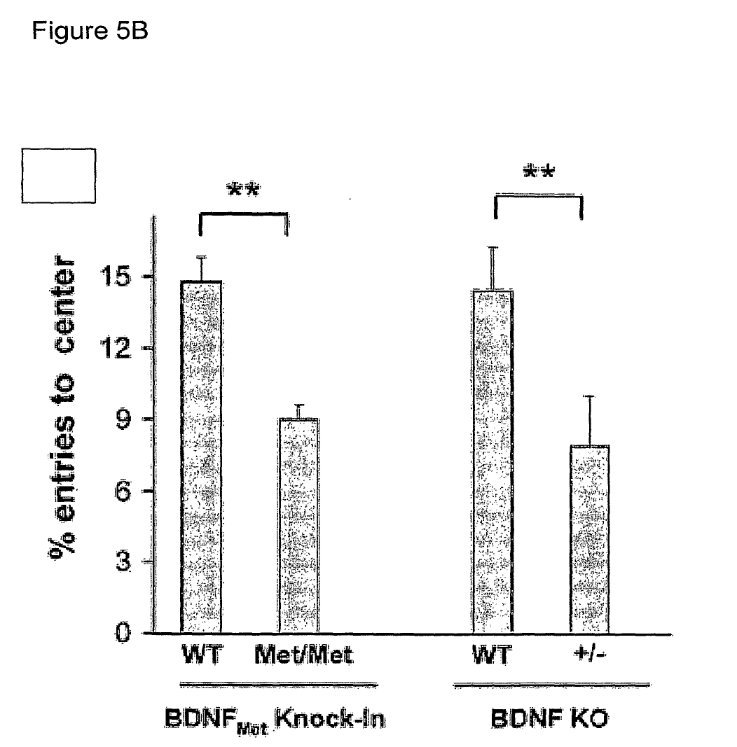

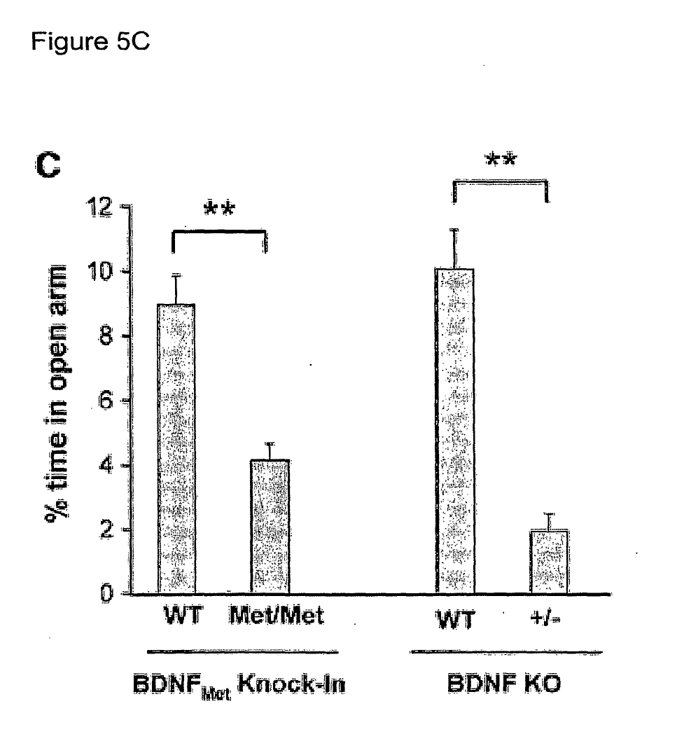

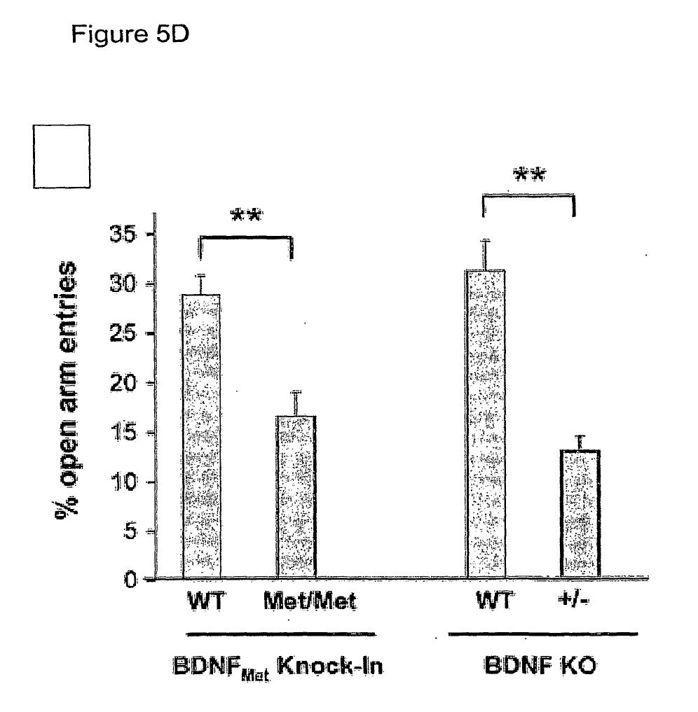

Two standard measures of anxiety-like behavior that place subjects in conflict situations were performed on adult BDNF_{Met} mice. In comparison with littermate wild-type control mice, BDNF^{Met/Met} mice had decreased exploratory behavior as demonstrated by a reduction in the percentage of time spent in the center compartment (FIG. 5A) and the number of entries into the center compartment (FIG. 5B) in the open-field test. BDNF^{Met/Met} mice also, exhibited, in the elevated plus maze test, a significant decrease in the percentage of time spent in open arms (FIG. 5C) and a significant reduction in the percentage of entries into open arms (FIG. 5D). In both tests, there were no significant differences in total distance traveled or the number of entries into enclosed arms between groups. In both of these tests, heterozygous BDNF^{+/Met} mice did not display increases in anxiety-related behaviors. BDNF^{+/-} mice also displayed increased anxiety-related behaviors in these two tests, similar in effect size to BDNF^{Met/Met} mice (FIG. 5).

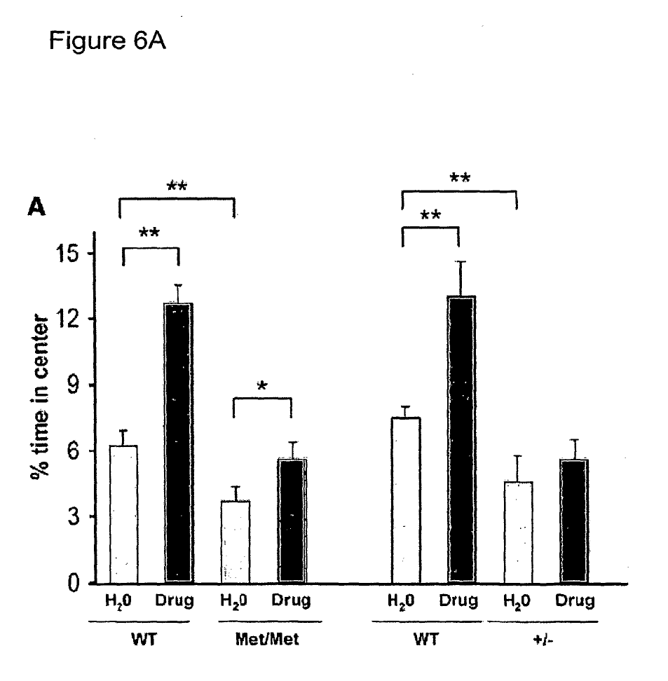

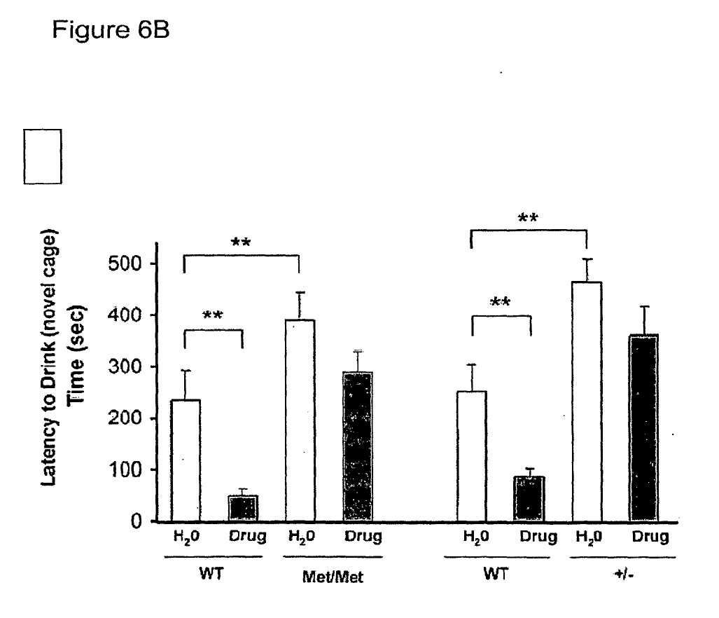

A common treatment for anxiety in humans are serotonin reuptake inhibitors (SSRIs). BDNF^{Met/Met} were treated orally with fluoxetine (18 mg/kg of body weight per day) or vehicle for 21 days before assessment in two tests: open field and novelty-induced hypophagia. In the open-filed test, fluoxetine led to a significant increase in time spent in the center for wild-type mice (FIG. 6A), as well as to an increase in entries into the center, which indicated its effectiveness in decreasing anxiety-related behaviors. However, there was a blunted response to fluoxetine in BDNF^{Met/Met} mice, with respect to time spent in the center (FIG. 6A), as well as entries into the center. Furthermore, the reduction in exploration could not be explained by changes in locomotor activity.

A specific behavioral paradigm for anxiety-related behavior, novelty-induced hypophagia, which has been suggested to be a more sensitive test of SSRI response was used. In this conflict test, mice are trained to approach a reward (e.g., sweetened milk) in their home cage and then placed in a novel brightly lit cage. The latency to approach and drink the sweetened milk is a measure of the anxiety-related behavior associated with this task. BDNF^{Met/Met} mice treated with vehicle has a significantly greater latency to drink in the novel cage as compared with wild-type controls (FIGS. 6B). Treatment with long-term fluoxetine did not significantly decrease the latency to drink in BDNF^{Met/Met} mice, as in wild-type littermate mice treated in parallel with long-term fluoxetine (FIG. 6B). In both of these assays, BDNF^{+/-} mice displayed similar diminished response to fluoxetine as compared with their wild-type controls (FIGS. 6A and 6B).

### Example 3

### Fluoxetine does not Reverse Anxiety-Related Behaviors in Transgenic Mice Homozygous for the G196A Polymorphism

A novel transgenic knock-in mouse expressing the variant BDNF (Val66Met, G196A) was generated in order to determine the role of this polymorphism on behaviors related to affective disorders. First, we confirmed that this transgenic mouse was a valid mouse model for the human variant BDNF polymorphism. Neurons cultured from these transgenic mice have significantly decreased BDNF secretion, as well as similar neuroanatomical defects as found in humans (decreased hippocampal volume).

Second, we determined that these mice have increased anxiety-related behaviors as assessed by 3 conflict/stress tests (open field, elevated plus maze, novelty induced hypophagia). These tests for anxiety differ from those used in human studies in that the animals' anxiety-related behaviors are assessed after placement in a stressful environment.

Third, we determined that chronic administration of a standard anti-anxiety, antidepressant medication, fluoxetine, could not reverse these anxiety-related behaviors in transgenic mice homozygous for the G196A polymorphism, while fluoxetine could robustly diminish anxiety-related behaviors in control wild-type mice.

1. A method for determining whether a patient suffering from a condition that is susceptible to treatment with a compound that activates the brain serotonin system is resistant to treatment with the compound, the method comprising:
(i) observing whether the genome of the patient contains at least one copy of the BDNF allele containing a genetic alteration, and
(ii) correlating the presence of the allele containing the genetic alteration with patients who are resistant to treatment with the compound.

2. The method of claim 1, wherein the genetic alteration comprises a frame shift.

3. The method of claim 1, wherein the genetic alteration comprises one or more nucleotide additions.

4. The method of claim 1, wherein the genetic alteration comprises one or more nucleotide deletions.

5. The method of claim 1, wherein the genetic alteration comprises one or more substitutions.

6. The method of claim 5, wherein the genetic alteration comprises a single nucleotide polymorphism.

7. The method of claim 6, wherein the single nucleotide polymorphism is G196A.

8. The method of claim 6, wherein the single nucleotide polymorphism is C5T.

9. The method of claim 6, wherein the single nucleotide polymorphism is G225T.

10. The method of claim 6, wherein the single nucleotide polymorphism is G374T.

11. The method of claim 6, wherein the single nucleotide polymorphism is G380T.

12. The method of claim 1, wherein the BDNF genotype is homozygous.

13. The method of claim 12, wherein the BDNF genotype is homozygous for G196A.

14. The method of claim 1, wherein the BDNF genotype is heterozygous.

15. The method of claim 1, wherein the BDNF genotype is heterozygous for G196A.

16. The method of claim 1, wherein the compound is a tricyclic antidepressant.

17. The method of claim 1, wherein the compound is an SSRI.

18. The method of claim 1, wherein the compound is an SNRI.

19. The method of claim 1, wherein the compound is an SARI.

20. The method of claim 1, wherein the condition is a psychiatric disorder.

21. The method of claim 1, wherein the psychiatric disorder is an affective disorder.

22. The method of claim 1, wherein the psychiatric disorder is an eating disorder.

23. The method of claim 1, wherein the eating disorder is bulimia.

24. The method of claim 1, further comprising prescribing to the patient a treatment other than administration of the compound.

25. The method of claim 1, further comprising administering to the patient a treatment other than administration of the compound.

26. A method for determining whether a patient suffering from a condition that is susceptible to treatment with a compound that activates the brain serotonin system is resistant to treatment with the compound, the method comprising:
(i) observing whether the patient expresses a BDNF protein containing an amino acid alteration, and
(ii) correlating the expression of the BDNF protein containing the amino acid alteration with patients who are resistant to treatment with the compound.

27. The method of claim 26, wherein the amino acid alteration comprises an amino acid substitution.

28. The method of claim 27, wherein the amino acid substitution is val66met.

29. The method of claim 27, wherein the amino acid substitution is thr2ile.

30. The method of claim 27, wherein the amino acid substitution is gln75his.

31. The method of claim 27, wherein the amino acid substitution is Arg125met.

32. The method of claim 27, wherein the amino acid substitution is arg127leu.
